(19) United States
(12) Patent Application Publication
(10) Pub. No.: US 2011/0269208 A1
(43) Pub. Date: Nov. 3, 2011

(54) CROSS-LINKED POLYMER NETWORK FORMED BY SEQUENTIAL CROSS-LINKING

(75) Inventors: Jason A. Burdick, Philadelphia, PA (US); Sudhir K. Khetan, Philadelphia, PA (US); Ross A. Marklein, Philadelphia, PA (US)

(73) Assignee: The Trustees of the university of Pennsylvania, Philadelphia, PA (US)

(21) Appl. No.: 13/059,125

(22) PCT Filed: Aug. 13, 2009
(86) PCT No.: PCT/US2009/053711

§ 371 Date: Jun. 2, 2011

Related U.S. Application Data
(60) Provisional application No. 61/089,092, filed on Aug. 15, 2008.

(51) Int. Cl. C12N 11/04

(52) U.S. Cl. 435/178

(57) ABSTRACT

The convention concerns methods for preparing a cross-linked polymer network comprising: forming a plurality of cross-links in a polymer network by (i) addition polymerization or (ii) ionic polymerization or (iii) partial radical polymerization of the polymer network to produce an intermediate polymer network, and forming further cross-links in the polymer network by radical polymerization of the intermediate polymer network to produce the cross-linked polymer network.

### CROSS REFERENCE TO RELATED APPLICATIONS

The present application claims benefit of U.S. Provisional Patent Application No. 61/089,092, filed Aug. 15, 2008, the disclosure of which is incorporated herein in its entirety.

### TECHNICAL FIELD

The invention concerns methods of forming cross-linked polymer networks, such as hydrogels, by sequential cross-linking

### BACKGROUND

The field of tissue engineering has expanded dramatically over the past decades due to the consistent need for novel therapies to treat the millions of patients each year that suffer from tissue and organ damage due to trauma or disease (Burg, et al, Biomaterials 2000; 21:2347-2359; Bonadio, Adv Drug Deliv Rev 2000; 44:185-194; and Elisseeff, et al., Orthod Craniofac Res 2005; 8:150-161). Much of the work in this field is motivated by the insufficient tissue available for transplantation (e.g., heart transplants) or the invasiveness of current techniques (e.g., autografts) (Goessler, et al., International Journal of Molecular Medicine 2005; 15:899-905; Vats, et al., Clinical Otolaryngology 2003; 28:165-172; Sun and Lal, Computer Methods and Programs in Biomedicine 2002; 67:85-103; and Bauer, Clinical Orthopaedics and Related Research 2000; 10-27). Typically, tissue regeneration involves the delivery of cells (e.g., primary cells, stem cells), stimulatory factors (e.g., gene therapy, growth factors), and a scaffolding material (e.g., porous sponges, hydrogels) (Vats, et al., Clinical Otolaryngology 2003; 28:165-172 and Vacanti and Bonassar, Clin Orthop Relat Res 1999, S375-381). The approach depends on numerous factors specific to the tissue, including the quantity and spatial distribution of cells, the regenerative capacity of the tissue, the local healing environment, and the importance and timing of functional restoration.

Stem cells are becoming a viable source of cells for tissue regeneration applications. Specifically, adult stem cells, which can be harvested directly from the patient, may provide an optimal autologous cell source for transplantation. One group of adult stem cells, mesenchymal stem cells (MSCs), are attractive since they can undergo extensive self-replication and differentiate into many cell types (Goessler, et al., International Journal of Molecular Medicine 2005; 15:899-905 and Caplan, Tissue Eng 2005; 11:1198-1211). MSCs may be isolated from a number of adult tissues, but the majority of work has focused on bone marrow-derived MSCs. Traditionally, the differentiation of MSCs has consisted of the introduction of stimulatory factors and control over the cell morphology (Goessler, et al., International Journal of Molecular Medicine 2005; 15:899-905 and Caplan, Tissue Eng 2005; 11:1198-1211). For instance, osteoblast differentiation occurs on two-dimensional (2D) surfaces in the presence of ascorbic acid and P-glycerophosphate, whereas chondrocyte differentiation occurs in pellets in the presence of TGF-Ps (Mauck, et al., Osteoarthritis and Cartilage 2006; 14:179-189 and Nuttelman, et al., Journal of Biomedical Materials Research Part A 2004; 68A:773-782). However, recent work has shown that MSC differentiation can be controlled through a variety of cues.

Certain of the factors that are important in controlling MSC differentiation and behavior in both 2D and 3D are outlined in FIG. 1. These include not only the introduction of soluble factors, but interactions with surrounding cells, substrate mechanics, biomaterial chemistry, surface-modification with factors that interact with cells, applied physical forces, and the degradation of the surrounding material (Alsberg, et al., Expert Opinion on Biological Therapy 2006; 6:847-866; Lensch, et al., Stem Cell Reviews 2006; 2:185-201; and Metallo, et al., Biotechnology Progress 2007; 23:18-23). Cells receive many signals from their surrounding environment through ligand-receptor interactions, so others have modified biomaterials with specific ligands to alter MSC behavior (Metallo, et al., Biotechnology Progress 2007; 23:18-23 and Nuttelman, et al., Matrix Biol 2005; 24:208-218). Others have also reported the importance of cell-shape on controlling stem cell differentiation (McBeath, et al., Developmental Cell 2004; 6:483-495).

It was recently reported (Engler, et al., Cell 2006; 126:677-689) that substrate elasticity (2D on polyacrylamide gels) directs stem cell lineage and that cells commit to phenotypes based on their tissue-specific elasticity (e.g., soft matrices are neurogenic, whereas more rigid matrices are osteogenic). The work was pioneering and important towards a better understanding of MSCs and controlling their differentiation. Also, it illustrated the importance of the cellular microenvironment, beyond soluble factors, on differentiation. In addition to differentiation, others have reported relationships between substrate compliance and cell behavior (Yeung, et al., Cell Motil Cytoskeleton 2005; 60:24-34; Engler, et al., Biophys J 2004; 86: 617-628; and Hammer, et al, Abstracts of Papers of the American Chemical Society 2005; 229:U648-U648). Cells also respond to gradients of mechanics, including durotaxis, or the migration of cells according to a mechanical gradient (Zaari, et al., Advanced Materials 2004; 16:2133-2137). However, the materials used in these prior investigations are limited in that viable cells can not be encapsulated in the gels to investigate the relevant 3D environment and that they require coating with extracellular matrix molecules (e.g., collagen) for cell adhesion.

Cellular spreading and attachment are key factors in tissue engineering. Cellular spreading is important in that it allows cells to interact with their environment, including receiving cues towards proliferation and even differentiation. Until recently, the scaffolding component in tissue engineering has been employed as a relatively inert component to the approach, providing mainly structural support and potential adhesion interactions through decoration with peptides and proteins. However, it is now clear that the dynamic interplay that occurs between cells and the extracellular matrix (ECM) is also important in the design and functionality of new biomaterials for use as synthetic cellular environments. The ECM is a dynamic and biologically active matrix with critical structural and functional roles, and ECM remodeling is necessary for cell migration and tissue morphogenesis.

Cellular spreading, which varies in vivo according to cell type and biochemical and mechanical properties of different tissues, may influence cellular functions such as stem cell differentiation. Past work indicates that hMSCs seeded onto substrates coated with adhesive elements such as fibronectin (Ogura, et al., J Oral Sci, 2004, 46, 207-213), collagen (Salasznyk, et al., J Biomed Biotechnol, 2004, 2004, 24-34, and gelaton (Shin, et al., Biomacromolecules, 2008, 9, 1772-1781) differentiate depending on adhesion, morphology, and spreading. Curran, et al. demonstrated that differences in morphology of hMSCs adhered to glass substrates with modified surface chemistries led to differences in differentiation. See, Curran, et al. and Curran, et al., Biomaterials, 2006, 27, 4783-4793. The importance of cell shape in terminal differentiation has also been demonstrated for other progenitor cell types ranging from bone marrow stromal cells to human epidermal keratinocytes.

Recent studies have incorporated ECM-mimetic features into hydrogels in a 3-dimensional (3-D) fashion to control encapsulated cellular behavior. For example, it has been shown that the viability and proliferation of hMSCs encapsulated in synthetic PEG-based hydrogel networks depends on the adhesiveness of the surrounding matrix (Salinas and Anseth, J Tissue Eng Regen Med, 2008, 2, 296-304). Beyond adhesion, Lutolf, et al. (Adv Mater., 2003, 15, 888-892) demonstrated that spreading and random migration of fibroblasts encapsulated in PEG-based hydrogels was possible when both cell-adhesivity and MMP-degradability were incorporated into the networks. They have since explored this system for cardiac tissue engineering applications (Kraehenbuehl, et al., Biomaterials, 2008, 29, 2757-2766), showing that multipotent cardioprogenitors encapsulated in the networks differentiated along the cardiac lineage when the stiffness of the scaffold mimicked that of native cardiac tissue. In a similar manner, Kim et al (J Mater Sci Mater Med., 2008, In Press) incorporated cell-adhesivity and proteolytic degradability into hyaluronic acid (HA) based scaffolds and demonstrated spreading of encapsulated hMSCs, something that was not possible in gels lacking either bioactive feature. Others have also utilized hydrogels containing these cues for tissue engineering approaches.

Despite these approaches, very few studies have investigated the spatial control that may be possible in these environments. In one example, investigators micropatterned cell-adhesive oligopeptides into precisely defined channels and demonstrated guided neurite outgrowth. (Luo and Shoichet, Nat Mater., 2004, 3, 249-253 and Musoke-Zawedde and Shoichet, Biomed Mater., 2006, 1, 162-169). However, the spatial control of encapsulated cell behavior using a cytocompatible process has not yet been achieved; the described 3-D studies all employed a single mode of crosslinking (e.g., addition reactions between peptide thiol groups and vinyl double bonds) homogeneously throughout the matrix volume, whereas the spatially controlled hydrogels did not use processes that were compatible in the presence of cells. A technique that affords such spatial control would be useful in numerous applications, from fundamental investigations of the influence of gel structure on cellular behavior to tools for advanced tissue engineering applications.

### SUMMARY

In some aspects, the invention concerns methods for preparing a cross-linked polymer network comprising:

forming a plurality of cross-links in a polymer network by (i) addition polymerization or (ii) ionic polymerization or (iii) partial radical polymerization of said polymer network to produce an intermediate polymer network, and

forming further cross-links in said polymer network by radical polymerization of said intermediate polymer network to produce said cross-linked polymer network.

In some embodiments, the polymer network is a hydrogel.

Suitable polymer networks include polysaccharide or any polymer with that contains reactive pendant groups (such as acrylates) along the backbone. Other useful polymers include polymers that contain charged moieties along the backbone (such as alginates). Useful hydrogels include acrylated (or methacrylated) hyaluronic acid or acrylated (or methacrylated) alginate. In certain methods, acrylated hyaluronic acid is 10-100 or, in some embodiments, 10-30 or 20-70 or 20-80, mole percent acrylate or methacrylate modified. Polymer networks of the invention can contain a plurality of biodegradable links such as caprolactone or MeLAHA moieties. Some compositions can contain enzymatically degradable cross-linkers. One such cross-linker is Ac-GCRD-GPQGIWGQ-DRCG-NH₂ (SEQ ID NO:1). Yet other compositions can contain adhesive peptides coupled to the gel prior to cross-linking. Such peptides include Ac-GCGYG-RGD-SPG-NH₂ (SEQ ID NO:2).

Some methods use a polymer network, such as a hydrogel, that comprises a plurality of cells. In some embodiments, the cells are in the polymer network prior to the cross-linking In some embodiments, the invention concerns methods of encapsulating cells comprising:

suspending the cells in a precursor solution which is gelled to form a hydrogel;

forming a plurality of cross-links in the hydrogel by (i) addition polymerization, (ii) ionic polymerization or (iii) partial radical polymerization of said hydrogel to produce an intermediate hydrogel, and

forming further cross-links in the hydrogel by radical polymerization of the intermediate hydrogel to produce the cross-linked hydrogel.

Preferred cells include embryonic stem cells or mesenchymal stem cells.

Some methods concern novel material-based processes that utilize multiple modes of crosslinking in a sequential manner to spatially control the behavior of cells encapsulated within 3-D hydrogels (one such process is shown schematically in FIG. 14). During the primary step, hydrogels that contain both adhesive sites and MMP-cleavable dithiol crosslinkers can be formed from multi-acrylate macromers (i.e., acrylated hyaluronic acid) via an addition mechanism, leaving a network that is “permissive” to remodeling and cellular spreading. Importantly, only a portion of the total number of acrylate groups is consumed during this first step, which occurs in the presence of a photoinitiator (at this point, non-reactive). During the secondary step, the gels are exposed to light to initiate radical polymerization of all the remaining acrylates, creating a network that is “inhibitory” to cell spreading based on the covalent crosslinks formed through kinetic chains. The premise is that the presence of covalent crosslinks blocks cellular remodeling and prevents cellular spreading in the hydrogels since the mesh sizes are significantly smaller than typical cell diameters. Since the second step is initiated by light, which can be spatially controlled, it is anticipated that this approach may be useful to spatially control cellular spreading within the hydrogels. Herein, we describe a process and its utility in controlling stem cell behavior in 3-D hydrogel environments.

Certain methods utilize a precursor solution further comprises a therapeutic agent. The resulting product, thus contains a therapeutic agent. Certain preferred therapeutic agents comprise gene therapy agents or growth factors.

Yet another aspect of the invention concerns methods of controlling stem cell differentiation comprising:

suspending the stem cells in a precursor solution which is gelled to form a hydrogel;

forming a plurality of cross-links in the hydrogel by (i) addition polymerization, (ii) ionic polymerization or (iii) partial radical polymerization of said hydrogel to produce an intermediate hydrogel, and

forming further cross-links in the hydrogel by radical polymerization of the intermediate hydrogel to produce the cross-linked hydrogel.

The invention also concerns products made by the methods described herein.

### BRIEF DESCRIPTION OF THE DRAWINGS

FIG. 1 depicts various factors important in cellular behavior in both 2-D and 3-D microenvironments.

FIG. 2 illustrates encapsulation of hESCs in HA or dextran gels with controlled differentiation.

FIG. 3 shows MSC staining for CD44 (A, inset for FACs analysis of CD44 on MSCs), the accumulation of type II collagen in PEG (B) versus HA (C) hydrogels, the expression of various genes by MSCs in HA gels normalized to PEG hydrogels (D), and a decrease in gene expression when cells are cultured in the presence of CD44 antibodies (E).

FIG. 4 illustrates the CS distribution by MSCs entrapped in gels of various MeHA:MeLAHA ratios.

FIG. 5 shows: Left: Mechanics (AFM) and imaging (fluorescent dye) of HA gels without (no UV) and with (UV) polymerization of a PEG IPN. Right: Imaging of various masks and patterns of increased crosslinking

FIG. 6 shows spreading in 3D gels using various cross-linking mechanisms. MMP gels allow spreading (with RGD present), whereas photopolymerized gels do not. Combinations can be used to spatially control spreading.

FIG. 7 presents a scheme for the sequential crosslinking of hydrogels using either Addition/Radical (top) or Ionic/Radical (bottom) mechanisms.

FIG. 8 shows the chemical structures of AHA (for Addition/Radical) and MA (for Ionic/Radical).

FIG. 9 presents a schematic of a technique to spatially control cell spreading in 3D hydrogels. Cell migration is only anticipated in regions not exposed to light.

FIG. 10 presents a schematic of a technique to spatially control hydrogel mechanics in 3D.

FIG. 11 presents characterization of sequentially crosslinked AHA hydrogels. (A) Compressive modulus and (B) swelling ratio of photopolymerized and sequentially crosslinked AHA hydrogels. The sequential crosslinking was performed with a theoretical consumption of either 50% or 75% of acrylates on the AHA during the primary crosslinking (C) Degradation kinetics of AHA hydrogels crosslinked using only an addition or radical mechanism (100% theoretical consumption of acrylates in both cases) in the presence of 40 nM MMP-1.

FIG. 12 presents hMSC encapsulation in sequentially crosslinked AHA hydrogels. (A) Images of encapsulated hMSCs (stained with calcein) in hydrogels formed by a photoinitiated polymerization alone, an addition polymerization alone, or using the sequential polymerization procedure. The sequential crosslinking was performed with a theoretical consumption of either 50 or 75% of acrylates on the AHA during the primary crosslinking Scale Bar=100 μm. (B) Histogram of the cellular aspect ratio (the ratio of the longest to shortest dimension of encapsulated cells) for these same groups. All cultures were for 5 days.

FIG. 13 presents spatially patterned outgrowth of hMSCs. (A) Schematic of process to spatially control cell spreading in AHA hydrogels. (B) Calcein stained hMSCs encapsulated in a sequentially crosslinked AHA hydrogel where one half of the construct was covered with a mask during light exposure. Scale Bar=100 μm. (C) Histogram of cell spreading in regions exposed to light (addition+radical) or covered with a mask (addition only) during the secondary crosslinking All cultures were for 5 days.

FIG. 14 show a schematic of sequential crosslinking of AHA using a primary addition reaction and a secondary radical polymerization. Adhesive Site (SEQ ID NO:2). Degradable Site (SEQ ID NO:1).

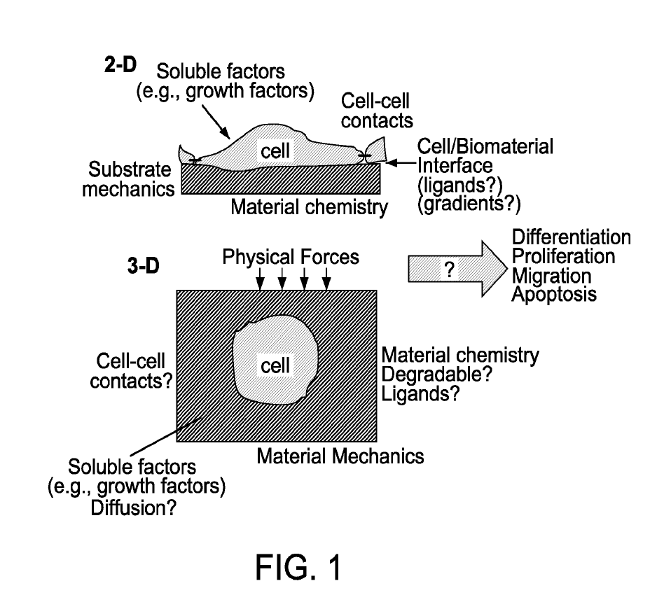

### DETAILED DESCRIPTION OF ILLUSTRATIVE EMBODIMENTS

Polymer networks (either highly crosslinked or loosely crosslinked as hydrogels) are useful for a wide range of applications and the control over bulk network properties is crucial. Typically, these properties are defined by parameters such as the crosslinking density or crosslinker concentration and are not dynamic in time or space. The instant invention overcomes these limitations by utilizing a sequential crosslinking procedure, where different modes of crosslinking are used on the same network. For example, a gel can be polymerized with an ionic mechanism to a low crosslinking and then further crosslinked (either in bulk or spatially, also with temporal variations if desired) through a radical mechanism (e.g., photo polymerization). Likewise, an addition polymerization can be used to form a network and then a radical polymerization again to further crosslink the networks.

The techniques described herein can be used for the control of cells in 3D networks. In one aspect, the invention focuses on the synthesis of novel hydrogel networks by sequential, multiple-mode crosslinking toward controlled cellular behavior in 3-dimensions. Individual crosslinking methods include addition (via Michael-Type reactivity), ionic, and radical (via addition of long wave UV light) polymerization. A typical application of the sequential crosslinking utilizes ionic or addition crosslinking in the first step, followed by radical photo polymerization in the second step. Patterning of features into the biomaterial is then afforded by application of a high-resolution photomask prior to the second polymerization, since only unmasked regions undergo biologically inert radical crosslinking. Because patterned regions in the hydrogels differ markedly in biochemical composition and mesh size, the technology can be used to manipulate the spatial behavior of encapsulated or invading cells or other particles (e.g. growth factors for drug delivery). The feature sizes attainable in the hydrogels are on the order of single microns with the use of 20,000 DPI masks, theoretically allowing for control of cellular behavior on the single-cell level. The technology also allows a practitioner to take advantage of extensive experience in functionalizing materials with reactive groups, e.g. acrylates for radical polymerization, through polymer synthesis. As such, the potential applications for the technology are very wide, as any polymeric material that can be chemically modified to contain reactive groups used in crosslinking can be used.

We have synthesized multiple acellular and cellular systems showing evidence of spatial patterning using the above process. The employment of the technology depends essentially independently upon the development of materials that can undergo the respective crosslinking reactions, the optimization of parameters (e.g., concentration of photoinitator) for the crosslinking reactions, and the development and application of the (optional) patterning element (i.e. photomasks in the systems explored thus far). Parameters can be optimized for individual applications and the invention produces an advance in controlling cell spreading and migration.

Potential commercial uses and applications include biological applications such as tissue engineering scaffolds. The controlled migration and behavior of cells in 3-dimensions is a critical skill for the regeneration of some tissues. For example, the synthesis of scaffolds with narrow biodegradable conduits has obvious potential for neural or cardiac engineering applications. Essentially, this could be platform technology for obtaining multiple cell types from one (e.g., stem cell differentiation through spatially controlled properties).

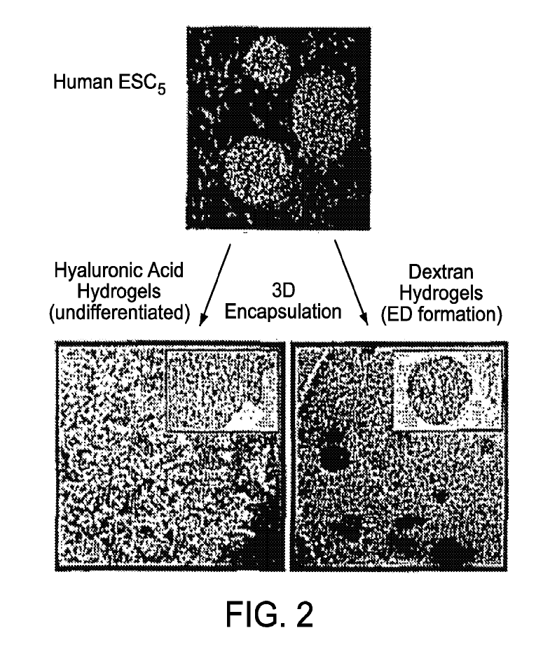

Control of self-renewal and differentiation of stem cells remains a challenge. This is largely due to the use of culture systems that involve poorly defined animal products, and do not mimic the normal developmental milieu. We recently developed hydrogels based on hyaluronic acid (HA) (Burdick, et al., Biomacromolecules 2005; 6:386-391 and Chung, et al., Journal of Biomedical Materials Research Part A 2006; 77A:518-525) and showed that this hydrogel matrix supports long-term self-renewal of human embryonic stem cells (hESCs) and directs cell differentiation without the use of animal products (Gerecht, et al., Acad Sci USA 2007; 104:11298-11303). HA hydrogels were selected because of the role of HA in early development and the controllability of hydrogel architecture, mechanics and degradation. When encapsulated in 3D HA hydrogels (but not within other hydrogels), hESCs maintained their undifferentiated state, preserved their normal karyotype, and maintained their full differentiation capacity. As shown in FIG. 2, hESCs remained undifferentiated in the HA gels, but spontaneously differentiated in gels fabricated from dextran, using similar biomolecule modification and encapsulation. However, controlled differentiation could be induced within the same hydrogel by simply altering soluble factors.

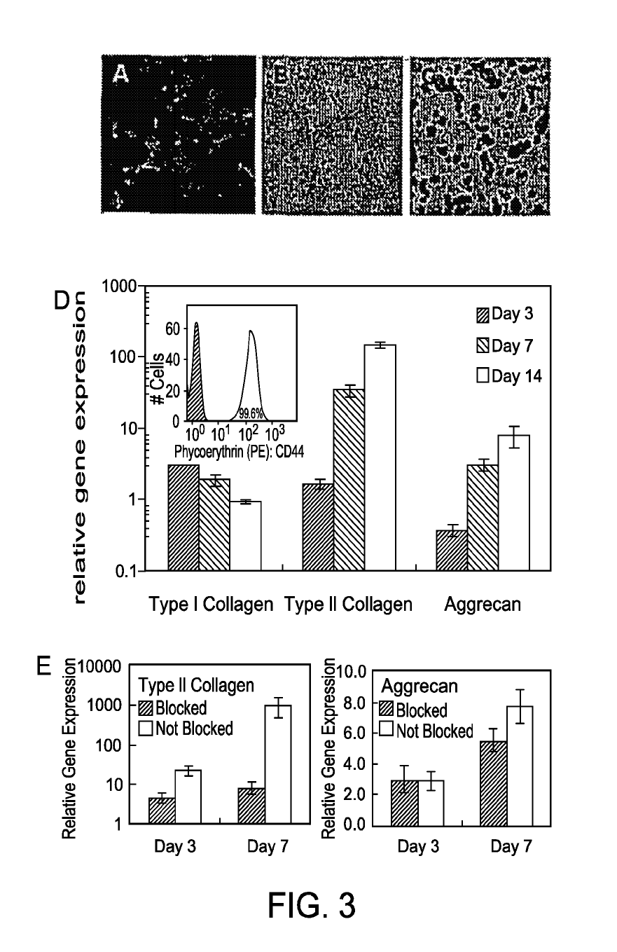

Towards our efforts in cartilage tissue engineering, we also encapsulated MSCs in HA gels (Chung C, Burdick JA. Influence of 3d hyaluronic acid microenvironments on mesenchymal stem cell chondrogenesis. Tissue Eng 2008; in press). The motivation was the abundance of CD44 receptors on the MSCs (FIG. 3A, inset), the presence of HA in native tissues, and the importance of HA in cellular processes, and thus, the likelihood of cell/hydrogel interactions. When MSCs were encapsulated in the HA gels, compared to inert PEG gels (that cells will not interact with), a significant upregulation in chondrogenic genes (type II collagen, aggrecan, FIG. 3D) was seen. Additionally, this led to greater ECM accumulation (FIG. 3B,C) in HA over PEG gels. Finally, when the CD44 receptors were blocked (FIG. 3E), this enhanced gene expression in HA gels was diminished.

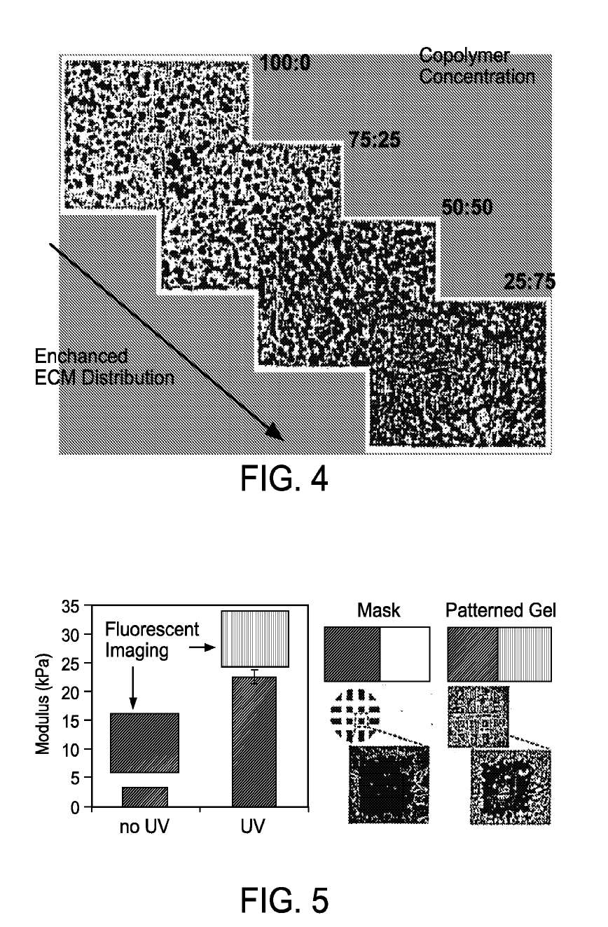

We have synthesized HA hydrogels that degrade via both enzymatic and hydrolytic means to allow for enhanced control over hydrogel properties (Sahoo, et al, Biomacromolecules 2008; 9:1088-1092). The HA macromer consists of degradable groups between the HA backbone and photoreactive methacrylate group. Alterations in the macromer concentration, number of degradable units, number of modified hydroxyl groups on HA, and type of degradable unit (caprolactone instead of lactic acid) can influence hydrogel degradation. These are readily modified during synthesis. The synthesized hydrolytically degradable macromer (MeLAHA with lactic acid) was copolymerized with the previously used enzymatically degradable MeHA at various ratios (100:0, 75:25, 50:50, 25:75, 0:100 MeHA:MeLAHA) and a concentration of about 2 wt %. MSCs remained viable (>95%) after encapsulation in all hydrogels and the cellular morphology changed depending on the copolymer concentrations. Notably, chondroitin sulfate (CS) distribution and macroporosity were observed with increasing inclusion of the hydrolytically degradable macromer (FIG. 4). Specifically, a gradient of CS (an important ECM molecule in cartilage) distribution corresponded with the amount of MeLAHA incorporated. This is an important finding and supports our ability to synthesize and modify hydrogels to alter MSC behavior.

Towards spatially controlling hydrogel properties, techniques to locally alter crosslinking in gels can be utilized. One approach involves crosslinking a hydrogel, swelling in a solution of an alternate macromer containing initiator, polymerizing with UV light to form interpenetrating networks (IPNs) with increased moduli, and swelling out unreacted macromer. As shown in FIG. 5, we are able to obtain gels (based on HA, with a poly(ethylene glycol) (PEG) IPN) where the elastic modulus (measured with atomic force microscopy) increases after light exposure and formation of the IPN. A methacrylated rhodamine dye was incorporated in the IPN solution (so it only stays where the secondary polymerization occurs and washes from all other areas), thus, it is easy to visualize where the secondary polymerization occurs. When light exposure occurs through a mask, patterning of the IPN is obtained and visualized fluorescently (FIG. 5). The magnified images show good correlation between mask features and crosslinking, although we are currently improving our mask printing abilities for better defined features. These tools (secondary polymerization, AFM, and dye for visualization of radical polymerization) can used to spatially control and assess hydrogel properties.

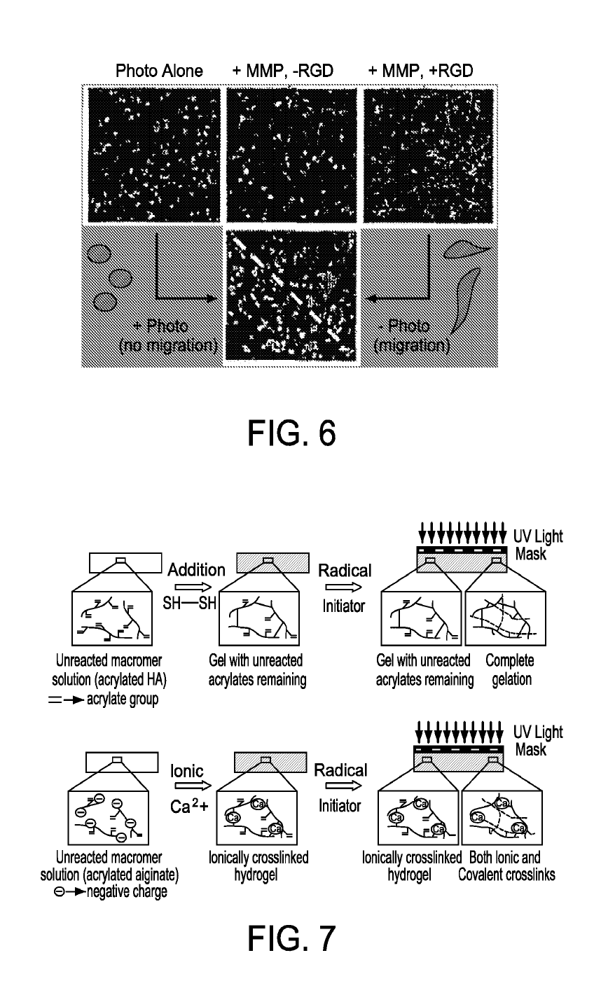

To overcome limitations with the lack of cellular spreading in many hydrogels, we have designed gels that incorporate multiple modes of crosslinking. We hypothesized that cell migration was inhibited in systems that contain only covalent crosslinks (inhibitory), yet cells would spread in systems that can be remodeled through MMP activity (permissive). We modified our HA hydrogels to support these behaviors and showed limited cell spreading in inhibitory environments and significant spreading in permissive environments (FIG. 6). Due to the unique material design, we were able to perform crosslinking sequentially to spatially control spreading by using masks to block light during the secondary polymerization (i.e., spreading occurs only where light was blocked). Although these results are preliminary, they are important towards our ability to control MSCs in 3D.

The environment surrounding stem cells may play an important role in dictating their behavior (i.e., spreading, migration, differentiation). Water-swollen polymer networks (hydrogels) are being developed as synthetic microenvironments for cells and are of interest due to the controllability of their properties (i.e., chemistry and mechanics) and their tissue-like nature. Although the hydrogels used in previous work by others (i.e., polyacrylamide) were important in addressing fundamental questions, there is little flexibility in controlling their temporal and spatial structure and due to monomer toxicity, it is not possible to encapsulate cells in 3D in these hydrogels. Thus, the instant invention's synthesis of novel multifunctional macromers that form hydrogel systems that can be exploited to further understand and control the spatial cellular environment in 3D on stem cell behavior. Importantly, this work develops sequential crosslinking procedures and assess gel properties.

Certain approaches are illustrated in FIG. 7. Hydrogels can be formed first using either: (1) a Michael-type addition reaction or (2) an ionic polymerization, and then further crosslinked spatially with UV light exposure through a mask using a radical polymerization of unreacted (meth)acrylate groups (i.e., increase crosslinking density). The use of these sequential polymerizations allows unique properties to be obtained using steps that are non-toxic to the encapsulation of MSCs. Addition (Lutolf, et al., Proc Natl Acad Sci USA 2003; 100:5413-5418 and Lutolf, et al, Nat Biotechnol 2005; 23:47-55), ionic (Kong, et al., Biomaterials 2003; 24:4023-4029; Alsberg, et al., Proceedings of the National Academy of Sciences of the United States of America 2002; 99:12025-12030; and Rowley, et al., Biomaterials 1999; 20:45-53) and 1 and radical (Temenoff, et al., Journal of Biomedical Materials Research Part A 2004; 70A:235-244 and Temenoff, et al., Biomacromolecules 2003; 4:1605-1613) polymerizations have been used successfully for cell encapsulation, but this may be the first attempt to utilize these mechanisms sequentially for spatial control over hydrogel properties with cells present.

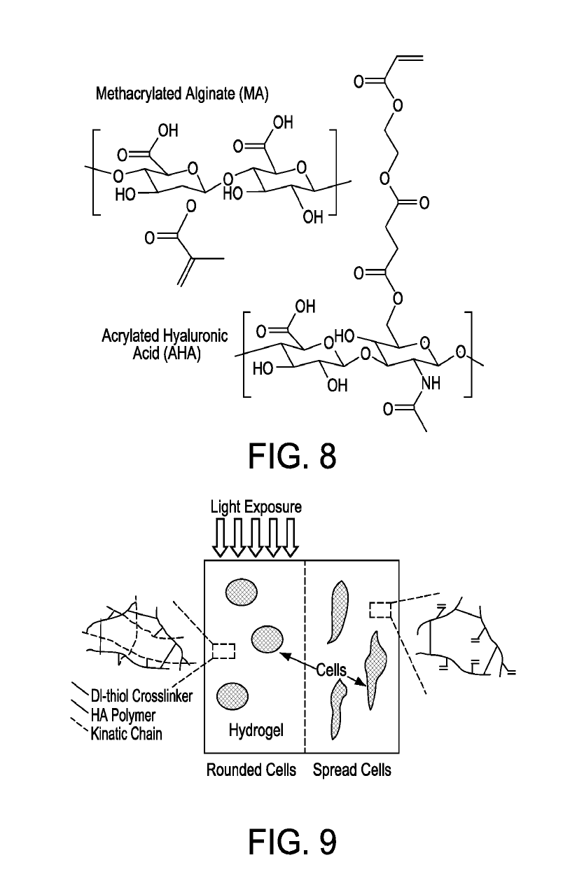

For the Addition/Radical reaction, an acrylated hyaluronic acid (AHA, FIG. 8) can be utilized with acrylate modifications of, for example, 10, 20, and 30%. Dithiothreitol (DTT) can be used as the initial crosslinker for this Objective. AHA (1, 2, 5, and 10 wt %) and DTT can be mixed at various ratios (with photoinitiator, constant at 0.05 wt %) for the addition reaction between the acrylate (electron-poor olefin) and thiol (nucleophile). The ratio between acrylate:thiol can be altered from 0.25:1, 0.5:1, 0.75:1, and 1.1. The presence of the photoinitiator allows for the secondary radical polymerization with unreacted acrylates and proceeds until full conversion of acrylates. It is expected that the acrylation %, AHA concentration, and variations in the acrylate:thiol will lead to changes in hydrogel mechanics.

For the Ionic/Radical reaction, a methacrylated alginate (MA, FIG. 8) can be utilized with % methacrylations of 10, 20, and 30%. The synthesis of the alginate is similar to the acrylation procedure for HA and we have synthesized methacrylated versions of alginate (Smeds, et al., J Biomed Mater Res 2001; 54:115-121). The MA (1, 2, 5, and 10 wt %) is mixed with various concentrations of calcium sulfate (1, 5, 10, 20 mg/ml) in the presence of a photoinitiator (constant at 0.05 wt %) for gelation and subsequently exposed to UV light for the secondary radical reaction. The methacrylation %, MA concentration, and variations in the calcium sulfate concentration can lead to changes in hydrogel mechanics.

The synthesized macromers can be characterized with ¹H-NMR and gel permeation chromatography (GPC, Waters). The hydrogel mechanics can be characterized with the range of parameters stated above (48 combinations for each sequential polymerization technique), both before and after the secondary radical polymerization using atomic force microscopy (AFM). The primary mechanical property of interest is the elastic modulus as determined with AFM and the target is a range of mechanics from about 1-100 kPa, to exploit information that is known about cellular mechanical responsiveness (Engler, et al., Cell 2006; 126:677-689 and Engler, et al., Biophys J 2004; 86:617-628). Finally, the viability of encapsulated MSCs is assessed, with and without sequential crosslinking in all compositions 1, 3, 5, and 7 days after encapsulation and with in vitro culture. Encapsulation is performed by simply mixing in the MSCs during the first step of the polymerizations. Although spatial patterning is not reported herein, radical polymerizations are commonly polymerized through masks and with lasers for spatial control (Khademhosseini, et al., Lab Chip 2004; 4:425-430 and Liu and Bhatia, Biomedical Microdevices 2002; 4:257-266).

Certain aspects of the invention are illustrated by the following non-limiting examples.

### Methods

### Synthesis of Acrylated HA (AHA) and Methacrylated Alginate (MA)

AHA is synthesized according to a 3-step protocol. First, hydroxyl ethyl acrylate (HEA) is reacted with succinic anhydride and 1-methylimidazole (65° C., 18 hrs, purified via extraction and MgSO₄ drying). Next, the tetrabutylammonium (TBA) salt of HA is synthesized by adding the acidic ion exchange resin Dowex-100 to a 1 wt % sodium-HA in deionized (DI) water (8 hrs), filtering the resin, neutralizing with 0.2M TBA-OH, and lyophilizing. Finally, HEA-Succinate is coupled to HA (equimolar, Argon) in the presence of 0.05 eq of dimethylamino pyridine (DMAP) catalyst. Anhydrous DMSO is cannulated into the vessel to dissolve the contents, and following heating to 45° C., 1 eq of ditertbutyl-dicarbonate is added and reacted overnight. The final product is dialyzed against DI water and lyophilized.

MA is synthesized by adding methacrylic anhydride to a 1 wt % solution of alginate (low viscosity, Sigma) in DI water, continuously adjusting the pH to 8 with 5N NaOH for 6 hours and then reacting for another 18 hours at 0° C. The macromer is purified by dialysis and lyophilization. The amount of modification is controlled by the amount of HEA for the AHA synthesis and amount of methacrylic anhydride for MA synthesis. The final products can be characterized with ¹H-NMR and GPC (Waters).

### Michael-Type Addition Reaction

AHA (various ratios) is dissolved in a triethanolamine-buffered saline (2M TEOA, 0.3M total osmolarity, pH 8.0), with 10% (v/v) of 0.5 wt % 12959/TEOA buffer solution. DTT is be dissolved in the same TEOA buffer and then added to this solution at various ratios. The solution is be mixed with repeated pipetting and then ejected into a mold (5 mm diameter, 2 mm height) and allowed to react for 15 minutes at room temperature. The gel is then be placed into PBS for further testing.

### Ionic Polymerization

MA (various ratios) is dissolved in PBS containing 0.05 wt % 12959. Calcium sulfate (Sigma) is dissolved in various concentrations in PBS, mixed with the MA solutions, and added to sterile molds (5 mm diameter, 2 mm height). The alginate is be allowed to crosslink for 15 minutes.

### Radical Polymerization

For studies that involve the secondary radical polymerization, the hydrogel (gelled with either addition or ionic polymerization) is placed between two glass slides and exposed to ultraviolet light (10 mW/cm) for 10 min with a long wave UV lamp encompassing a collimating lens (EXFO). Our previous work indicates that light exposure at these intensities does not compromise membrane integrity, alter mitochondrial activity, increase the production of p53 in response to light-induced DNA damage, or result in altered DNA (Gerecht, et al., Proc Natl Acad Sci USA 2007; 104:11298-11303; Bryant, et al., J Biomater Sci Polym Ed 2000; 11:439-457; and Wang, et al., Tissue Eng 2005; 11:201-213), and thus, no issues related to light exposure are expected.

### Mechanical Testing

Upon gelation, hydrogels are swollen in PBS for 24 hours and then tested using AFM. An AFM with a sphere-tipped cantilever (spring constant 60 pN/nm) and blunted AFM tips (half-open angle of 35° C.) are used to indent the hydrogels (2 μm/s). Force measurements can be performed to obtain displacement curves that are be fitted with a Hertz model to obtain the elastic modulus.

### Cell Encapsulation and Viability

Human MSCs (Cambrex) is passaged using standard culture conditions (Caplan, Tissue Eng 2005; 11:1198-1211; Baksh, et al., Journal of Cellular and Molecular Medicine 2004; 8:301-316; and Short, et al., Archives of Medical Research 2003; 34:565-571) and encapsulated in the hydrogels by suspending cells (5 million/ml) in the precursor solutions during the first gelation steps. The macromers and solutions are sterilized by either exposing to a germicidal lamp for 15 minutes or by filter sterilization when possible. The viability of entrapped cells can be monitored with both a Live/Dead assay kit (Molecular Probes) and an MTT mitochondrial activity assay (Sigma) immediately after gelation and after 1, 3, 5, and 7 days of in vitro culture (DMEM+20% FBS+pen/strep). Viability is assessed in all possible hydrogel variations (48 total for each sequential polymerization process).

### Statistical Analysis

Analysis of variance (ANOVA) is carried out using the STATISTICA software package (Statsoft, Tulsa, Okla.), with Tukey's HSD post-hoc testing of differences between groups with α=0.05.

### Use of Hydrogels to Control Spreading and Migration of MSCs

In pioneering work, West and Hubbell (West, et al., Macromolecules 1999; 32:241-244) developed synthetic polymer networks that mimicked key components of the native ECM, including enzymatically degradable crosslinks and adhesive ligands. Further work has been performed using this general concept (Mann, et al., Biomaterials 2001; 22:3045-3051; Gobin and West, Faseb Journal 2002; 16:-; and Nguyen and West, Biomaterials 2002; 23:4307-4314) and it has been shown that both enzymatically degradable groups and adhesive ligands are necessary for cell spreading and migration in 3D. These biomimetic structures are important in that they can be engineered to control cell outgrowth and migration rates through the incorporation of specific crosslinks. However, there is little spatial control in these previous systems. To overcome this limitation, we use a system of multiple crosslinks that can be designed for spatial control over cell spreading. Cell spreading/migration is inhibited in systems that contain covalent crosslinks (Bryant, et al., Biomaterials 2001; 22:619-626; Burdick, et al., Biomaterials 2002; 23:4315-4323; and Elisseeff, et al., Journal of Biomedical Materials Research 2000; 51:164-171), yet cells will spread where there is only enzymatically degradable crosslinks. Thus, through control over light exposure (with masks), cell spreading can be controlled (FIG. 9). Cell spreading is important to control since it is variable depending on the cell type and location and because spreading can lead to differences in cellular differentiation (Senechal, et al., In Vitro 1984; 20:692-698 and Shin, et al., Biomacromolecules 2008; 9:1772-1781). Thus, control over spreading, particularly spatially, allows future control over cellular constructs.

To our knowledge, there are currently no synthetic materials available that provide this spatial control over cell spreading, using a cytocompatible process (i.e., performed in the presence of cells, not seeded with cells after modification). We have performed experiments on a material that allows for cellular spreading and through sequential crosslinking can be controlled spatially. In this approach, cells are encapsulated in a material that is permissive to spreading (contains MMP degradable crosslinkers, adhesion sites) and then hinder spreading in areas by adding covalent crosslinks via a radical photoinitiated polymerization through unreacted acrylate groups. This allows cells to spread in the permissive areas and remain rounded in the inhibitory areas. These hydrogels are formed with the Addition/Radical mechanisms described herein illustrate the potential to control spreading and migration in the gels, and to develop specific correlations between the gel structure and cell behavior. The materials to be used include the AHA materials, with the addition of thiol containing enzymatically degradable peptide crosslinkers (e.g., Ac-GCRD-GPQGIWGQ-DRCG-NH₂) (SEQ ID NO:1) and adhesive peptides (Ac-GCGYG-RGD-SPG-NH₂) (SEQ ID NO:2) (Lutolf, et al, Proc Natl Acad Sci USA 2003; 100:5413-5418 and Lutolf and Hubbell, Nat Biotechnol 2005; 23:47-55).

Studies can be performed first without patterning to illustrate the general idea that sequential crosslinking can inhibit spreading. The amount of addition versus radical polymerization through the ratio of acrylate to thiol during the addition polymerization can be investigated, as well as AHA concentration and % acrylation. The groups are determined as those that supported gelation and the viability of entrapped cells (>90% after 7 days). First, hydrogel degradation are monitored (released uronic acid) both with hyaluronidase (50 U/ml) and MMP-2 (40 nM). Gels that do not degrade in the presence of MMP-2 prior to radical polymerization are not be used further. Second, cells are encapsulated in the hydrogels (5 million/ml) and assessed for viability (Live/Dead) and spreading (NIH image analysis of confocal images) after 2, 5, and 10 days. Groups will be without and with the secondary radical polymerization and the adhesive peptide concentration is maintained constant. If too much enzymatically degradable crosslinker is used, there is not be enough radical polymerization to inhibit migration and that if not enough enzymatically degradable crosslinker is used, the hydrogels will be resorbed too rapidly.

Next, spatial patterning is be performed using groups that showed spreading with only the addition mechanism and no spreading when the secondary radical polymerization is used. Masks can be used to spatially control light exposure for radical polymerization with a variety of patterns and gradients. Since the light is collimated, the features can be defined throughout the gels. The patterns are consist first of covering one-half of the gel with a mask. When this is performed successfully, masks that encompass circular “dark” areas ranging from 100-1000 um, with distances between features of 100-1000 um. Also, linear gradients can be used that go from completely “dark” to completely “light” over lengths of 1-10 mm. The patterned gels can be characterized by mechanics (AFM), as well as copolymerization with a methacrylated dye (50 uM methacryloxyethyl thiocarbamoyl rhodamine B). The dye is only incorporated into the network during the radical polymerization, so it gives a visual indication of the spatial hydrogel properties, so confocal microscopy can be used to determine the definition of the patterns with depth. Once the hydrogel patterning is assessed, MSCs can be incorporated and the gels and spatial spreading can be assessed in all of the different patterns using live/dead staining and confocal imaging. Again, spreading can be quantified using NIH image analysis of confocal images.

Controlling cell migration is also essential. To assess migration in these hydrogels a simple assay can be used where cells are encapsulated in a hydrogel (only those permitting spreading will be utilized) and then an acellular hydrogel of the same composition can be polymerized to the surface of the first gel. The distance that the cell front migrates across and into the acellular gel can be monitored at 1, 3, 5, and 7 days and quantified using live/dead staining and confocal imaging. This work can lead to correlations between hydrogel chemistry and cell migration. Once this is completed, the acellular hydrogel can be replaced with gels of the same patterns and gradients as above to assess migration into specific patterns and whether the mechanical gradients will permit and induce migration.

### Peptide Synthesis

The enzymatically and adhesive peptides used can be custom synthesized through a facility at University of Pennsylvania.

### Hydrogel Formation

The RGDS-containing cell-adhesive peptide is dissolved in TEOA buffer and added to a final concentration in the AHA/photoinitiator solution of 1 mM, and allowed to react for 30 minutes at 37° C. The MMP peptide in TEOA buffer is be used in place of DTT and added for the addition gelation step.

### Hydrogel Degradation

Formed hydrogels are placed in PBS and incubated alone or with the addition of either 50 U/ml of hyaluronidase or 40 nM MMP-2. Samples are taken intermittently throughout degradation and assessed for uronic acid, using a common procedure (Burdick, et al., Biomacromolecules 2005; 6:386-391 and Sahoo, et al., Biomacromolecules 2008; 9:1088-1092).

### Confocal Microscopy

Cells are stained with Live/Dead or gels are polymerized with the rhodamine dye (Polysciences) and assessed using standard techniques with a confocal microscope (Ar—Kr laser, available in Bioengineering Confocal and Multiphoton Imaging Core Facility). NIH Image can be used to measure the aspect ratio of cells to quantify spreading.

### Spatial Patterning

Common techniques for the fabrication of masks can be used (Chen, et al, Mrs Bulletin 2005; 30:194-201; Ostuni, et al., Langmuir 2000; 16:7811-7819; and Chen, et al., Biotechnology Progress 1998; 14:356-363) and a variety of masks of gradients and islands (both as the light-blocking and the light-permeable areas) can be made. Masks can be printed on transparencies using an inkject printer. The mask can be placed directly on the hydrogel for patterning through exposure to high intensity light (EXFO lamp, collimating lens adaptor) for intermittent periods.

### MSC Differentiation in 3D Hydrogels Through Spatially Controlled Crosslinking Density

Substrate mechanics are able to dictate cellular differentiation and can be exploited using a cell-compatible hydrogel material (described herein) that allows for analysis in 3D. The first step towards analysis is seeding the MSCs on the surface of adhesive peptide modified alginate gels with a range of mechanics (ranging from about 1-100 kPa elastic modulus). This essentially replicatse what was previously performed, but with our new hydrogel system rather than the collagen modified acrylamide gels. The importance of this new hydrogel system is that the adhesive peptide can be coupled to the alginate prior to crosslinking and that cells can be encapsulated in the hydrogels in 3D. This allows for decoupling of all cellular interactions with the material beyond mechanical properties. Immunostaining can be used initially to monitor differentiation. In some embodiments, the alginate system is used, rather than the HA system, so that spreading/migration and potential HA interactions are not factors in the cellular interactions, but only matrix mechanical properties. Cells (seeded at 5 or 20 thousand cells per cm²) can be assessed for up to 7 days in 2D. Based on previous reports (Engler, et al, Cell 2006; 126:677-689), osteogenic, myogenic, and neurogenic differentiation can be investigated with CBFAI, MyoD, and P3-tubulin as the initial markers, respectively. In addition to these common immunostaining markers, it is expected that additional markers can be identified through the use of gene arrays. Also, contraction force microscopy (Hammer, et al., Abstracts of Papers of the American Chemical Society 2005; 229:U648-U648 and Reinhart-King, et al., Langmuir 2003; 19: 1573-1579) can be utilized to better understand and quantify the interactions of cells with the surfaces of these hydrogels.

Once 2D studies are completed, the cells can be encapsulated in these same gels in 3D. To accomplish this, cells can be encapsulated in alginate gels with a wide range of mechanics (about 1-100 kPa) and then either cultured as is or exposed to light sequentially to increase the entire gel mechanics. Specific formulations can be identified and those that induced lineage specific differentiation in the 2D studies. These constructs can be cultured in standard growth media and assessed for up to 7 days as above with immunostaining and real time rt-per. Comparisons can be made between the 2D and 3D experiments.

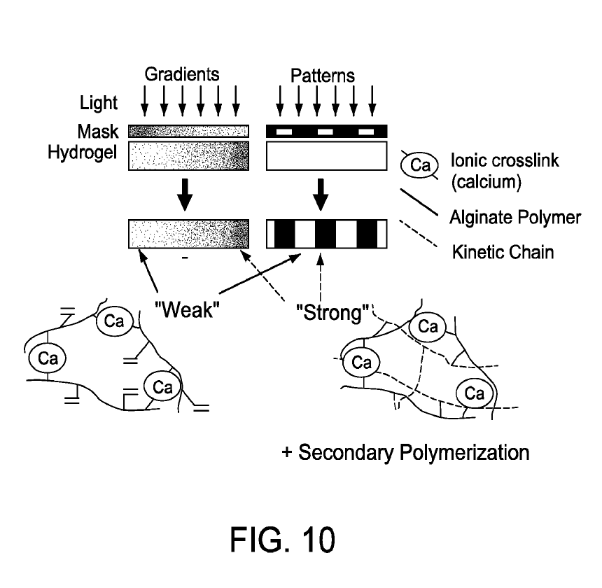

Using this information and towards the development of multi-cellular constructs, the spatial patterning of hydrogels (i.e., mechanics) can also be investigated. The work of others (Dillmore, et al., Langmuir 2004; 20:7223-7231) and our previous work (Burdick, et al., Langmuir 2004; 20:5153-5156) showed that patterns and gradients of adhesive ligands are possible on polymeric microstructures. Alterations in mechanical properties have also been shown to influence cellular durotaxis (i.e., migration due to gradient of mechanics) in 2D (Zaari, et al., Advanced Materials 2004; 16:2133-2137). However, there are no reports on the effects of substrate gradients and patterns on MSC differentiation, specifically in 3D. Yet, spatially controlling cellular differentiation could play a major role in advancing both basic biology and the development of novel approaches towards the engineering of complex tissues. To accomplish this, hydrogels can be fabricated as just described, yet the sequential crosslinking can be performed by exposure of these formed hydrogels to light through a variety of masks with either patterned areas or gradients that block light (FIG. 10). Again, ligands can be incorporated into the gels to control adhesion and the ligand density is not expected to change with light exposure (beyond changes with swelling).

With patterning techniques, “islands” of a “weak” hydrogel (about 1 kPa) can be placed in a “strong” hydrogel (about 100 kPa) and vice versa. The size of the “island” can be systematically varied (100-1000 pm diameters) to assess the potential of obtaining multi-phenotype differentiation using local mechanics and how spatial differentiation is influenced by “island” size. The patterned materials can be thoroughly characterized through AFM. After fabrication and characterization of gradient and patterned substrates, spatial alterations in differentiation can be monitored with a wide range of mechanical gradients (based on results from above project) and changes in differentiation can be correlated with the gradient magnitude. Immunostaining can be performed as the primary outcome measure of differentiation.

### Immunostaining/Gene Arrays

Standard techniques can be used for immunostaining Cells can be fixed in 2.5% glutaraldehyde, blocked with serum, permeabilized with Triton-X, and labeled with CBFAI (Alpha Diagnostic International), MyoD (Chemicon), and p3-tubulin (Sigma) primary antibodies and secondary antibodies for visualization. Nuclei can be stained with DAPI. For gene expression analysis, total RNA can be obtained from the cells cultured on the gels using a Trizol reagent (Sigma) and can be hybridized to custom oligonucleotide arrays. Gene expression can be normalized to MSCs seeded onto or encapsulated within the gels. Microarrays can be performed through the Microarray core with the Penn Center for Musculoskeletal Disorders.

### Traction Force Microscopy

This technique can be conducted as described previously (Hammer, et al., Abstracts of Papers of the American Chemical Society 2005; 229:U648-U648 and Reinhart-King, et al., Langmuir 2003; 19: 1573-1 579). Traction forces can be determined from deformations of 0.5 pm beads embedded in the gels. Intermittently, phase contrast (of cells) and fluorescent (of beads) images can be taken, as well as an image of the beads in their unstressed state. Using custom software, the bead displacements can be calculated and traction vectors determined. This can be performed on gels initially and with exposure to light.

The invention is further illustrated by the following non-limiting examples whose methods and results are reported herein.

AHA synthesis. Acrylated hyaluronic acid (AHA) was synthesized via a 3-step protocol. All ¹H NMR spectra were recorded on a Bruker Avance 360 MHz spectrometer.

Synthesis of HEA-Succinate (HEA-suc). Succinic anhydride (1.5 eq) and 2-hydroxyethyl acrylate (1 eq) were combined in a 500 mL three neck round bottomed flask. Following a purge of nitrogen, 200 mL of anhydrous dichloroethane was cannualted into a flask and the reaction was heated to 65° C. 1-methylimidazole was added as a catalyst (0.06 eq). The reaction was allowed to proceed for 18 hours at 65° C. The product was purified by extractions with aqueous 0.1M HCl and 1M NaCl and the organic layer was dried with MgSO4. To avoid polymerization, a small amount of hydroquinone was added prior to drying. NMR (CDCl₃): δ 2.70 ppm, 4H, m; 4.37 ppm, 4H, m; 5.87 ppm, 1H, dd; 6.14 ppm, 1H, dd; 6.44 ppm, 1H, dd.

Synthesis of the tetrabutylammonium salt of HA (HA-TBA). Sodium hyaluronate (1 eq) was dissolved in 200 mL DI H₂O to give a ˜1 wt % solution. To this solution, the highly acidic ion exchange resin, Dowex-100 (3 eq, by mass), was added, and the slurry was stirred for eight hours, at which point the solution was filtered to remove the resin. The acidic solution was neutralized with 0.2M tetrabutylammonium hydroxide (TBA-OH) to pH 7.02-7.05, forming a quaternary ammonium salt of hyaluronate and the tetrabutylammonium group (HA-TBA). The solution was frozen and lyophilized to yield the dry product. NMR (D₂O): δ 4.2-4.6 ppm, 2H; 3.15-3.9 ppm, 10H; 3.1 ppm, 8H, dd; 1.9 ppm, 3H; 1.5 ppm, 8H; 0.82 ppm, 12H.

Coupling of HEA-suc and HA-TBA. HA-TBA (1 eq, repeat unit) and dimethylamino pyridine (DMAP; 0.075 eq) were combined in an oven-dried 3-neck round-bottomed flask under nitrogen. The amount of DMAP and HEA-suc added relative to HA-TBA was varied to achieve different percentage acrylate functionalities. Anhydrous DMSO was cannulated into the sealed flask to give a roughly 1 wt % HA-TBA solution. The flask was heated to 45° C., and following complete dissolution of the contents, ditertbutyl-dicarbonate (1.5 eq) was injected into the flask and the reaction was allowed to proceed for 18 hours. The solution was then diluted 1:1 with DI H₂O, dialyzed extensively against DI H₂O, frozen, and lyophilized to yield the dry product.

The final structure and ¹H NMR spectrum of AHA: NMR (D₂O): δ 6.4 ppm, 0.4H, d; 6.15 ppm, 0.4H, dd; 5.9 ppm, 0.4H, d; 4.2-4.6 ppm, 2H; 3.15-4.0 ppm, 10H; 2.7 ppm, 1.2H, broad; 1.9 ppm, 3H, s.

### Cells

Human mesenchymal stem cells (hMSCs) were obtained from Lonza Corporation (Wakersville, Md.). For encapsulation studies, hMSCs were expanded in growth media (a-MEM, 10% FBS, 1% L-Glutamine & penicillin-streptomycin) and encapsulated at low passage numbers (between 2 and 4) in AHA hydrogels at a density of 5×10⁶ cells mL⁻¹. The constructs were maintained in 1.5 mL of growth media in a 24-well plate and refreshed every three days until the end of day 5, at which point live/dead analysis was performed.

### Peptides

The cell adhesive oligopeptide GCGYGRGDSPG (SEQ ID NO:2, MW: 1025.1 Da) and MMP-degradable oligopeptide GCRDGPQG(ARROW)IWGQDRCG (SEQ ID NO:1, MW: 1754.0 Da), both with >95% purity (per manufacturer HPLC analysis), were obtained from GenScript Corporation (Piscataway, N.J., USA) for all studies.

### Crosslinking

AHA was dissolved in a triethanolamine-buffered saline (TEOA buffer: 0.2M TEOA, 0.3M total osmolarity, pH 8.0) containing Irgacure® 2959 (Ciba, “12959”) photoinitiator (final concentration of 0.05 wt %). 12959 was chosen due to its aqueous solubility and good cytocompatibility. The cell adhesive peptide dissolved in TEOA buffer was added to the AHA solution at a final peptide concentration of 1 mM (corresponding to ˜ 1/20th of available acrylate groups with 3 wt % AHA), and allowed to react for one hour at 37° C. Following re-suspension of cells in this solution, MMP peptide dissolved TEOA buffer was added to the pre-polymer solution corresponding to the desired percent acrylate consumption, and 50 μL of this mixture was immediately pipetted into sterile molds (5 mm diameter, 2 mm height). The gels were allowed to react (primary crosslinking) for 15 minutes at room temperature inside the laminar flow hood. For sequential crosslinking studies, gels were then exposed to 10 mW cm⁻² 365 nm ultraviolet light (Omnicure S1000 UV Spot Cure System, Exfo Life Sciences Division, Mississauga, Ontario, Canada) for 4 minutes (secondary crosslinking). Gelation times were chosen based on earlier acellular experiments that measured the addition and radical polymerization durations (15 and 4 min, respectively) for which further reaction did not change the mechanical properties.

### Hydrogel Characterization

Acellular samples were fabricated as described above. Following crosslinking and swelling to equilibrium in PBS for 24 hours, the Young's modulus of each hydrogel disk was determined by unconfined submersion compression testing on a dynamic mechanical analyzer (Q800 Series; TA Instruments, New Castle, Del., USA) with an oscillating plate compression clamp attachment. Briefly, the diameter of each swelled hydrogel disk (˜5 mm) was determined using a digital caliper, and the sample was immersed in a PBS bath between unconfined parallel compression plates to prevent dehydration. An equilibrium preload force was applied by the descending plate, followed by application of a ramped strain of 10%/min to 60%. The Young's modulus was then determined using the slope of the stress-strain curve at low strain (<25% strain). To obtain the volumetric swelling ratio (QV), equilibrium swelled constructs were patted dry to remove surface liquid and weighed (wet weight), lyophilized, and re-weighed (dry weight). QV is reported as the ratio of the wet weight to dry weight, assuming a density of 1.23 g cm⁻³ for the AHA macromers. For degradation studies, hydrogels crosslinked completely (i.e., 100% acrylate consumption) through addition or radical polymerization were incubated in separate wells of a 24-well plate containing 1 mL PBS with 40 nM human MMP-1 (Sigma) on an orbital shaker at 37° C. The solutions were refreshed every 24 hours for one week, and the supernatant samples (frozen and stored at −20° after collection) were analyzed in triplicate via a modified uronic acid assay. Briefly, 100 μL of each sample was added to a concentrated solution of sulfuric acid/sodium tetraborate decahydrate (Sigma) and heated to 100° C. for 10 min. 100 μL of 0.125% carbazole solution in ethanol was then added, and the samples were vortexed and heated to 100° C. for 15 min. The sample absorbances were then read at 530 nm and compared to a standard curve of known concentrations of HA (a range from 0.1 to 2.0 mg mL⁻¹). All studies were performed in triplicate unless otherwise noted.

### Live/Dead Staining

Encapsulated cells were visualized for viability using a fluorescent live/dead staining kit (Molecular Probes) and imaging on an inverted microscope (Axiovert 200, Carl Zeiss Inc.) equipped with an epifluorescent lamp. For assessment of viability, three random images of each gel at 5× magnification were taken through both the live (FITC) and the dead (TRITC) filters. Cell viability was then assessed by counting the total number of live and dead cells in each image and calculating the ratio of live/total cells.

### Cellular Aspect Ratio Measurements

For cellular aspect ratio measurements, three random light microscopy images at 5× magnification were taken from each gel using an inverted microscope. To quantify cellular spreading, the maximum orthogonal length and width of each cell was measured using NIH ImageJ and the aspect ratio calculated as the longer length divided by the shorter length. Each image produced ≧15 measurements, or n≧45 for each sample. The measured aspect ratios were then sorted into bins to form histograms of spreading for each formulation.

### Acellular Hydrogel Synthesis and Characterization

AHA with ˜40% of hydroxy groups modified with acrylates was synthesized as described. HA, a linear glycosaminoglycan made of alternating D-glucuronic acid and N-acetyl-D-glucosamine, was used as the primary structural component due to its biocompatibility, hydrophilicity, importance in vivo including in the turnover of ECM following tissue injury, interactions with cells via surface receptors, and past use in tissue engineering applications. Although HA is commonly modified with methacrylate groups, acrylate groups were used since they react much faster during the primary addition step, which allows for uniform cell suspensions. While HA was used in the current work, the sequential crosslinking technique can be applied to any macromer functionalized with reactive groups that can undergo multiple modes of crosslinking, highlighting its versatility.

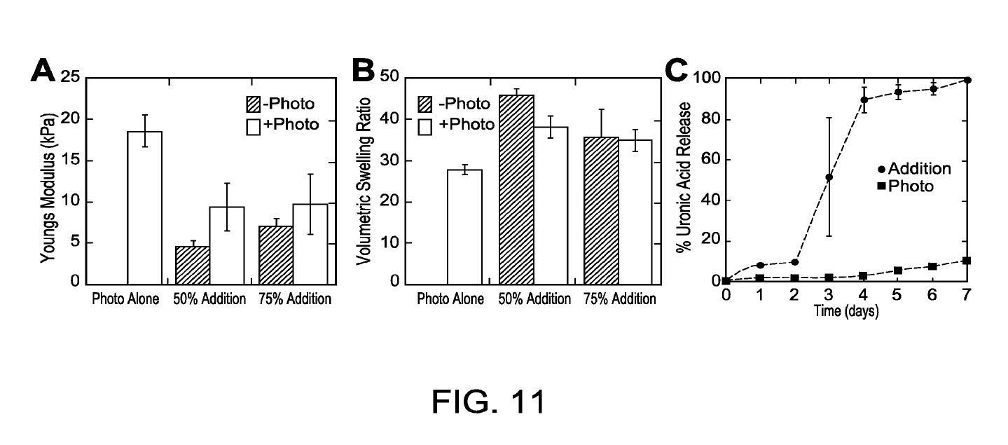

AHA was crosslinked into 3 wt % hydrogels either with a photoinitiated polymerization alone, with an addition polymerization alone, or sequentially using both (in order) an addition and radical polymerization. For the addition polymerizations, theoretically 50, 75, or 100% of the acrylates were consumed. Both the mechanics and the swelling of the hydrogels were dependent on the type of crosslinking (and for sequentially crosslinked gels, the ratio of addition to radical crosslinking) that was used (FIGS. 11A and 11B). Hydrogels crosslinked only through radical polymerization exhibited a ˜2-4 fold higher compressive modulus (18.62+/−1.96 kPa) and swelled significantly less (QV=27.75+/−1.20) then either addition alone (e.g., modulus=4.60+/−0.71 kPa, QV=45.42+/−1.70 for 50% formulation) or sequentially crosslinked (e.g., modulus=9.45+/−2.90 kPa, QV=38.26+/−2.68 for 50%+photo formulation) hydrogels. The kinetic chains in the radically crosslinked only hydrogels concentrate the acrylate side chains and create a more dense network than those that are reacted with a di-thiol oligopeptide crosslinker molecule, as in the addition reaction. This could explain the higher modulus and decreased swelling in radical only hydrogels. Sequentially crosslinked hydrogels with identical peptide compositions but secondarily crosslinked through photopolymerization exhibited increased moduli and decreased swelling relative to their addition-only counterparts, indicative of the secondary radical polymerization. These changes were greater for the 50% case, since a higher percentage of acrylate groups was available for the radical crosslinking step.

AHA hydrogels synthesized completely (i.e., 100% acrylate consumption) through the addition or radical crosslinking mechanisms also differed predictably in degradation kinetics when incubated in PBS containing 40 nM MMP-1 (FIG. 11C). Hydrogels crosslinked with MMP-degradable oligopeptides underwent complete degradation via the action of exogenous proteases by day 7, while radically crosslinked gels underwent comparatively little degradation (˜10% mass loss) with kinetics that mimicked incubation in PBS alone. These results support the underlying premise that the covalent kinetic chains do not allow for proteolytic degradation, whereas the MMP cleavable crosslinks degrade rapidly in the presence of the enzyme. These trends also illustrate the tunability of the sequential crosslinking system, as both the concentration of MMP-degradable domains (i.e., the degradation kinetics of the hydrogel in the presence of MMP) and bulk mechanical properties can be matched to the tissue engineering application of interest.

### Controlled Encapsulated Cell Spreading in Bulk Polymerized Gels

To determine if these results translate into cellular instructive hydrogels, hMSCs were suspended in the initial macromer solution and encapsulated using either the photoinitiated polymerization alone or the sequential crosslinking procedure.

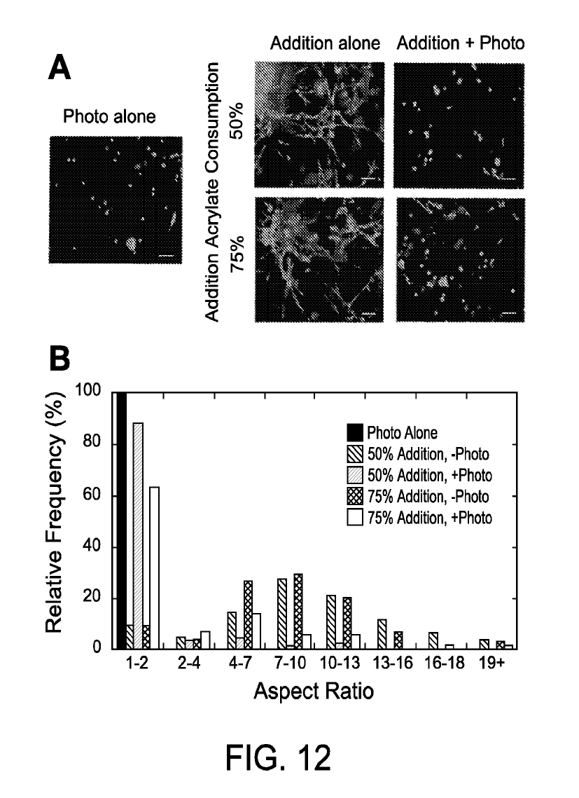

The addition alone hydrogels with 100% acrylate consumption polymerized too quickly to obtain evenly distributed cells and were not further investigated. As seen in FIG. 12, the mode of crosslinking dictated the spreading of encapsulated cells. As expected, hMSCs in constructs containing RGD but crosslinked only through radical polymerization remained rounded (FIG. 12A), with 100% of the cells exhibiting an aspect ratio (i.e., the ratio of the longest to shortest dimension of encapsulated cells) between 1 and 2 (FIG. 12B). In contrast, cells encapsulated in “permissive” hydrogels synthesized only through addition crosslinking using MMP-degradable peptides corresponding to 50% and 75% acrylate consumption exhibited relatively high degrees of spreading (i.e., a distribution towards much higher aspect ratios). However, cells encapsulated in “inhibitory” hydrogels formed with the sequential crosslinking procedure were similar to the radical polymerization alone and remained rounded.

This inhibition was more pronounced with a lower fraction of acrylates consumed during the addition step (50% versus 75%), potentially due to the greater amount of crosslinking during the secondary radical polymerization to inhibit spreading.

Cells in all conditions exhibited high viability (>90%) as quantified from live/dead staining (dead stain overlayed on FIG. 12 live images). Additionally, there was no spreading (aspect ratios from 1-2) for control hydrogels that did not include the adhesive peptide or for hydrogels that were crosslinked with an alternate di-thiol (DTT) that is not MMP-cleavable (results not shown). This indicates that both adhesion and degradation sites are necessary for cellular remodeling of synthetic hydrogels.

### Spatially Controlled Encapsulated Cell Spreading

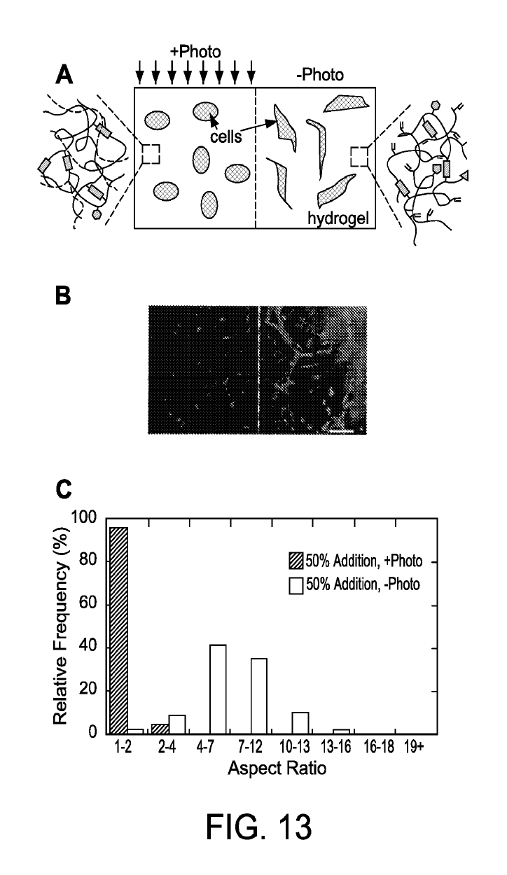

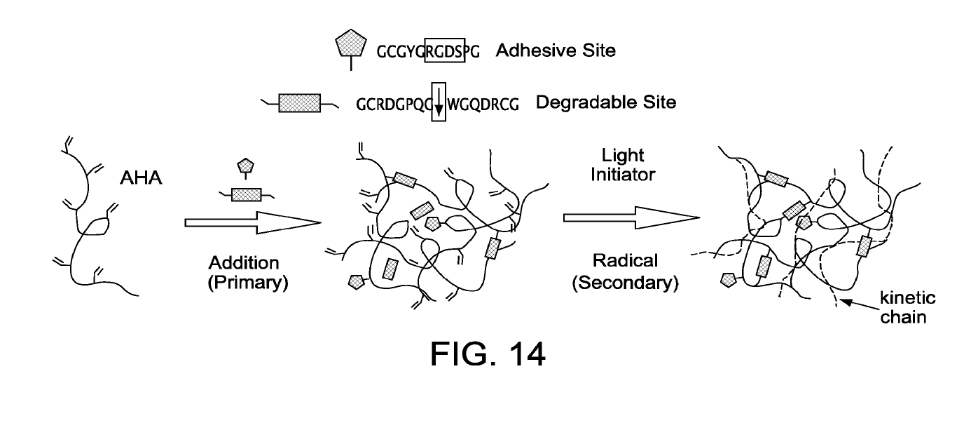

Although these results illustrate our ability to form gels that either permit or inhibit cell spreading, there are many instances where this would be beneficial to achieve with spatial control. As discussed, it is clear that cues such as spreading lead to changes in cell signaling and potentially differentiation; thus, spatial control over spreading could lead to control over cell lineage towards the development of advanced tissue engineering approaches with differentiation down multiple cell lineages. In this sequential crosslinking approach, the creation of spatially controlled spreading of AHA hydrogels can be achieved by applying a photomask between the two crosslinking steps (FIG. 13A). As illustrated, regions of the hydrogel that are unmasked are exposed to light and undergo a secondary radical polymerization, while masked regions are not exposed to the light and only undergo the primary crosslinking. To illustrate the feasibility of this approach, AHA hydrogels synthesized with 50% consumption during the primary crosslinking were exposed to light through a mask that blocked half of the sample in entirety. A live image of cells at the interface in this gel is shown in FIG. 13B and indicates spherical morphology with light exposure and spindle-like morphology in areas that were covered with the mask. The extent of outgrowth in these regions, both qualitatively from light microscopy and as quantified through aspect ratio measurements (FIG. 13C), were similar to the corresponding bulk gels assessed above. Although this is a simple example of the approach, more complex patterns could be achieved with different masks or through the use of lasers for the secondary polymerization.

The sequential polymerization described here is a robust, novel approach towards dictating the cellular behavior in 3-D. While a single AHA weight percentage was used in the current study, the versatility of the sequential crosslinking technology arises from the ability to vary this and other design parameters (e.g., HA acrylation efficiency, macromer and peptide concentrations, encapsulated cell density) to tune the remodeling kinetics to different applications. For instance, differences in cellular morphology in patterned AHA hydrogels could be useful as a signaling mechanism for spatially controlled differentiation of encapsulated stem cells. Such an approach has potential in the regeneration of tissues with anisotropic properties (e.g., vasculature or nervous tissues) or where spatially controlled organization of cells is desired. In the current work, the cells were cultured in standard growth medium to illustrate the technique of controlled spreading, and no specific cell type or tissue was targeted. Collectively, this approach may become a valuable tool in biomaterials development and regenerative medicine.

What is claimed:

1. A method for preparing a cross-linked polymer network comprising:
forming a plurality of cross-links in a polymer network by (i) addition polymerization, (ii) ionic polymerization or (iii) partial radical polymerization of said polymer network to produce an intermediate polymer network, and
forming further cross-links in said polymer network by radical polymerization of said intermediate polymer network to produce said cross-linked polymer network.

2. The method of claim 1, wherein the polymer network is a hydrogel.

3. The method of claim 2, wherein said hydrogel is (i) acrylated or methacrylated hyaluronic acid or (ii) methacrylated or acrylated alginate.

4. The method of claim 2, wherein said cross-linked hydrogel has a plurality of biodegradable links.

5. The method of claim 4, wherein said biodegradable links comprise caprolactone or MeLAHA moieties.

6. The method of claim 2, wherein said cross-linked hydrogel comprises an enzymatically degradable cross-linker.

7. The method of claim 6, wherein said enzymatically degradable cross-linker comprises Ac-GCRD-GPQGIWGQ-DRCG-NH₂ (SEQ ID NO:1).

8. The method of claim 2, wherein said hydrogel comprises an adhesive peptide coupled to said gel prior to cross-linking

9. The method of claim 8, wherein said adhesive peptide is Ac-GCGYG-RGD-SPG-NH₂ (SEQ ID NO:2).

10. The method of claim 2, wherein said hydrogel comprises a plurality of cells prior to said cross-linking

11. A method of encapsulation of cells comprising:
suspending said cells in a precursor solution which is gelled to form a hydrogel;
forming a plurality of cross-links in said hydrogel by (i) addition polymerization, (ii) ionic polymerization or (iii) partial radical polymerization of said hydrogel to produce an intermediate hydrogel, and
forming further cross-links in said hydrogel by radical polymerization of said intermediate hydrogel to produce said cross-linked hydrogel.

12. The method of claim 11, wherein said cells are embryonic stem cells or mesenchymal stem cells.

13. The method of claim 11, wherein said precursor solution further comprises a therapeutic agent.

14. The method of claim 11, wherein said therapeutic agent comprises a gene therapy agent or a growth factor.

15. The method of claim 11, wherein said hydrogel is acrylated hyaluronic acid or methacrylated alginate.

16. A method of controlling stem cell differentiation comprising:
suspending said stem cells in a precursor solution which is gelled to form a hydrogel;
forming a plurality of cross-links in said hydrogel by (i) addition polymerization, (ii) ionic polymerization or (iii) partial radical polymerization of said hydrogel to produce an intermediate hydrogel, and
forming further cross-links in said hydrogel by radical polymerization of said intermediate hydrogel to produce said cross-linked hydrogel.

17. The method of claim 16, wherein said hydrogel is acrylated hyaluronic acid or methacrylated alginate.

18. The method of claim 16, wherein said hydrogel comprises methacrylated alginate and contains an adhesive peptide.

19. The method of claim 16, wherein said adhesive peptide is Ac-GCGYG-RGD-SPG-NH₂ (SEQ ID NO:2).

20. The product of claim 11.


SEQUENCE LISTING (2 sequences)

SEQ ID NO: 1 - protein, 16 aa, Artificial Sequence
  Synthetic construct
GCRDGPQGIWGQDRCG

SEQ ID NO: 2 - protein, 11 aa, Artificial Sequence
  Synthetic construct
GCGYGRGDSPG